SEQUENCE LISTING (25 sequences)

SEQ ID NO: 1 - protein, 1273 aa, Artificial Sequence
  Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 19 aa, Artificial Sequence
  Severe acute respiratory syndrome coronavirus 2
EGFNCYFPLQSYGFQPTNG

SEQ ID NO: 3 - protein, 1255 aa, Artificial Sequence
  Severe acute respiratory syndrome-related coronavirus (SARS-CoV)
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCAFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 4 - protein, 198 aa, Artificial Sequence
  Severe acute respiratory syndrome-related coronavirus (SARS-CoV)
RFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCG

SEQ ID NO: 5 - protein, 19 aa, Artificial Sequence
  Severe acute respiratory syndrome-related coronavirus (SARS-CoV)
PALNCYWPLNDYGFYTTTG

SEQ ID NO: 6 - protein, 1353 aa, Artificial Sequence
  Middle East respiratory syndrome-related coronavirus
MIHSVFLLMFLLTPTESYVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYESFDVESGVYSVSSFEAKPSGSVVEQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKLEFANDTKIASQLGNCVEYSLYGVSGRGVFQNCTAVGVRQQRFVYDAYQNLVGYYSDDGNYYCLRACVSVPVSVIYDKETKTHATLFGSVACEHISSTMSQYSRSTRSMLKRRDSTYGPLQTPVGCVLGLVNSSLFVEDCKLPLGQSLCALPDTPSTLTPRSVRSVPGEMRLASIAFNHPIQVDQLNSSYFKLSIPTNFSFGVTQEYIQTTIQKVTVDCKQYVCNGFQKCEQLLREYGQFCSKINQALHGANLRQDDSVRNLFASVKSSQSSPIIPGFGGDFNLTLLEPVSISTGSRSARSAIEDLLFDKVTIADPGYMQGYDDCMQQGPASARDLICAQYVAGYKVLPPLMDVNMEAAYTSSLLGSIAGVGWTAGLSSFAAIPFAQSIFYRLNGVGITQQVLSENQKLIANKFNQALGAMQTGFTTTNEAFQKVQDAVNNNAQALSKLASELSNTFGAISASIGDIIQRLDVLEQDAQIDRLINGRLTTLNAFVAQQLVRSESAALSAQLAKDKVNECVKAQSKRSGFCGQGTHIVSFVVNAPNGLYFMHVGYYPSNHIEVVSAYGLCDAANPTNCIAPVNGYFIKTNNTRIVDEWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYYNKWPWYIWLGFIAGLVALALCVFFILCCTGCGTNCMGKLKCNRCCDRYEEYDLEPHKVHVH

SEQ ID NO: 7 - protein, 483 aa, Human immunodeficiency virus
SATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKR

SEQ ID NO: 8 - protein, 876 aa, Human immunodeficiency virus
MRVRGTLRNYQQWWIWGVLGFWMLMICNGGGKVEKDLWVTVYYGVPVWKEAKTTLFCASDAKAYDTEVHNVWATHACVPTDPNPQEMVLGNVTENFNMWKNEMVDQMQEDVISLWDQSLKPCVKLTPLCVTLECKNVSKHNASVTNSTLSGTYNESGQEIKNCTFNVTTELRDKKKKEYALFYRLDIVPLDEENSSNNSTSNNSSGNYRLINCNTSAITQACPKVNFDPIPIHYCTPAGYAILKCNNKTFNGTGPCNNVSTVQCTHGIKPVVSTQLLLNGSLAEEEIIIRSENLTDNVKTIIVHLNKPVEIECIRPNNNTRKSIRIGPGQTFYATGEIIGDIRQAYCIINASQWNETLYNVSKKLAERFNNRTINFTSSSGGDLEITTHSFNCRGEFFYCNTSRLFNSTYMSNGTYTYTINSNISNITIPCRIKQIVNMWQEVGRAMYAPPIAGNITCKSNITGLLLVRDGGSNETQPEIFRPGGGDMRDNWRSELYKYKVVEIKPLGLAPTTAKRRVVEREKRAVGIGAVFLGFLGAAGSTMGAASITLTVQARQLLSGIVQQQSNLLKAIEAQQHLLQLTVWGIKQLQTRVLAIERYLKDQQLLGIWGCSGRLICTTAVPWNSSWSNKTQKEIWDNMTWMEWDREINNYTDTIYRLLEESQNQQEKNEKDLLALDSWNNLWNWFNITNWLWYIRIFIMIVGGLIGLRIIFAVLSVVNRVRQGYSPLSFQIHTPNPGGPDRLGRIEEEGGEQDKNRSVRLVSGFLALVWDDLRNLCLFSYHQLRDFILVTARVVELLGRSSLRGLQKGWEALKYLGSLVQYWGLELKKSAISLFDSIAIAVAEGTDRIIEFIRRICRAIRNIPRRIRQGFEIALQ

SEQ ID NO: 9 - protein, 566 aa, Artificial Sequence
  Influenza A virus H1N1
  misc_feature (470)..(470): Xaa can be any naturally occurring amino acid
MKAILVVLLYTFAAANADTLCIGYHANNSTDTVDTVLEKNVTVTHSVNLLEDKHNGKLCKLRGVAPLHLGKCNIAGWILGNPECESLSTASSWSYIVETSSSDNGTCYPGDFIDYEELREQLSSVSSFERFEIFPKTSSWPNHDSNKGVTAACPHAGAKSFYKNLIWLVKKGNSYPKLSKSYINDKRKEVLVLWGIHHPSTTADQQSLYQNADAYVFVGTSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQXKNNAKEIGNGCFEFYHKCDNMCMESVKNGTYDYPKYSEEAKLNREEIDGVKLESTRIYQILAIYSTVASSLVLVVSLGAISFWMCSNGSLQCRICI

SEQ ID NO: 10 - protein, 469 aa, Artificial Sequence
  Influenza A virus H1N1
MNPNQKIITIGSVCMTIGMANLILQIGNIISIWISHSIQLGNQNQIETCNQSVITYENNTWVNQTYVNISNTNFAAGQPVVSVKLAGNSSLCPVSGWAIYSKDNSIRIGSKGDVFVIREPFISCSPLECRTFFLTQGALLNDKHSNGTIKDRSPYRTLMSCPIGEVPSPYNSRFESVAWSASACHDGINWLTIGISGPDNGAVAVLKYNGIITDTIKSWRNNILRTQESECACVNGSCFTIMTDGPSDGQASYKIFRIEKGKIVKSVEMNAPNYHYEECSCYPDSSEITCVCRDNWHGSNRPWVSFNQNLEYQIGYICSRIFGDNPRPNDKTGSCGPVSSNGANGVKGFSFKYGNGVWIGRTKSISSRKGFEMIWDPNGWTGTDNNFSIKQDIVGINEWSGYSGSFVQHPELTGLDCIRPCFWVELIRGRPKENTIWTSGSSISFCGVNSDTVGWSWPDGAELPFTIDK

SEQ ID NO: 11 - protein, 389 aa, Hepatitis B virus
MGQNLSTSNPLGFFPDHQLDPAFRANTANPDWDFNPNKDTWPDANKVGAGAFGLGFTPPHGGLLGWSPQAQGILQTLPANPPPASTNRQSGRQPTPLSPPLRNTHPQAMQWNSTTFHQTLQDPRVRGLYFPAGGSSSGTVNPVLTTASPLSSIFSRIGDPALNMENITSGFLGPLLVLQAGFFLLTRILTIPQSLDSWWTSLNFLGGTTVCLGQNSQSPTSNHSPTSCPPTCPGYRWMCLRRFIIFLFILLLCLIFLLVLLDYQGMLPVCPLIPGSSTTSTGPCRTCMTTAQGTSMYPSCCCTKPSDGNCTCIPIPSSWAFGKFLWEWASARFSWLSLLVPFVQWFVGLSPTVWLSVIWMMWYWGPSLYSILSPFLPLLPIFFCLWVYI

SEQ ID NO: 12 - protein, 192 aa, Hepatitis C virus
AEVKNISTGYMVTNDCTNDSITWQLQAAVLHVPGCVPCEKVGNASQCWIPVSPNVAVQRPGALTQGLRTHIDMVVMSATLCSALYVGDLCGGVMLAAQMFIVSPQHHWFVQDCNCSIYPGTITGHRMAWDMMMNWSPTATMILAYAMRVPEVIIDIISGAHWGVMFGLAYFSMQGAWAKVVVILLLAAGVDA

SEQ ID NO: 13 - protein, 367 aa, Hepatitis C virus
RTHTVGGSAAQTTGRLTSLFDMGPRQKIQLVNTNGSWHINRTALNCNDSLHTGFIASLFYTHSFNSSGCPERMSACRSIEAFRVGWGALQYEDNVTNPEDMRPYCWHYPPRQCGVVSAKTVCGPVYCFTPSPVVVGTTDRLGAPTYTWGENETDVFLLNSTRPPLGSWFGCTWMNSSGYTKTCGAPPCRTRADFNASTDLLCPTDCFRKHPDTTYLKCGSGPWLTPRCLIDYPYRLWHYPCTVNYTIFKIRMYVGGVEHRLTAACNFTRGDRCNLEDRDRSQLSPLLHSTTEWAILPCSYSDLPALSTGLLHLHQNIVDVQFMYGLSPALTKYIVRWEWVILLFLLLADARVCACLWMLILLGQAEA

SEQ ID NO: 14 - protein, 907 aa, Artificial Sequence
  Human gammaherpesvirus 4
MEAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPESTTTSPTLNTTGFADPNTTTGLPSSTHVPTNLTAPASTGPTVSTADVTSPTPAGTTSGASPVTPSPSPWDNGTESKAPDMTSSTSPVTTPTPNATSPTPAVTTPTPNATSPTPAVTTPTPNATSPTLGKTSPTSAVTTPTPNATSPTLGKTSPTSAVTTPTPNATSPTLGKTSPTSAVTTPTPNATGPTVGETSPQANATNHTLGGTSPTPVVTSQPKNATSAVTTGQHNITSSSTSSMSLRPSSNPETLSPSTSDNSTSHMPLLTSAHPTGGENITQVTPASISTHHVSTSSPAPRPGTTSQASGPGNSSTSTKPGEVNVTKGTPPQNATSPQAPSGQKTAVPTVTSTGGKANSTTGGKHTTGHGARTSTEPTTDYGGDSTTPRPRYNATTYLPPSTSSKLRPRWTFTSPPVTTAQATVPVPPTSQPRFSNLSMLVLQWASLAVLTLLLLLVMADCAFRRNLSTSHTYTTPPYDDAETYV

SEQ ID NO: 15 - protein, 706 aa, Artificial Sequence
  Human gammaherpesvirus 4
MQLLCVFCLVLLWEVGAASLSEVKLHLDIEGHASHYTIPWTELMAKVPGLSPEALWREANVTEDLASMLNRYKLIYKTSGTLGIALAEPVDIPAVSEGSMQVDASKVHPGVISGLNSPACMLSAPLEKQLFYYIGTMLPNTRPHSYVFYQLRCHLSYVALSINGDKFQYTGAMTSKFLMGTYKRVTEKGDEHVLSLVFGKTKDLPDLRGPFSYPSLTSAQSGDYSLVIVTTFVHYANFHNYFVPNLKDMFSRAVTMTAASYARYVLQKLVLLEMKGGCREPELDTETLTTMFEVSVAFFKVGHAVGETGNGCVDLRWLAKSFFELTVLKDIIGICYGATVKGMQSYGLERLAAMLMATVKMEELGHLTTEKQEYALRLATVGYPKAGVYSGLIGGATSVLLSAYNRHPLFQPLHTVMRETLFIGSHVVLRELRLNVTTQGPNLALYQLLSTALCSALEIGEVLRGLALGTESGLFSPCYLSLRFDLTRDKLLSMAPQEATLDQAAVSNAVDGFLGRLSLEREDRDAWHLPAYKCVDRLDKVLMIIPLINVTFIISSDREVRGSALYEASTTYLSSSLFLSPVIMNKCSQGAVAGEPRQIPKIQNFTRTQKSCIFCGFALLSYDEKEGLETTTYITSQEVQNSILSSNYFDFDNLHVHYLLLTTNGTVMEIAGLYEERAHVVLAIILYFIAFALGIFLVHKIVMFFL

SEQ ID NO: 16 - protein, 137 aa, Artificial Sequence
  Human gammaherpesvirus 4
MRAVGVFLAICLVTIFVLPTWGNWAYPCCHVTQLRAQHLLALENISDIYLVSNQTCDGFSLASLNSPKNGSNQLVISRCANGLNVVSFFISILKRSSSALTGHLRELLTTLETLYGSFSVEDLFGANLNRYAWHRGG

SEQ ID NO: 17 - protein, 223 aa, Artificial Sequence
  Human gammaherpesvirus 4
MVSFKQVRVPLFTAIALVIVLLLAYFLPPRVRGGGRVAAAAITWVPKPNVEVWPVDPPPPVNFNKTAEQEYGDKEVKLPHWTPTLHTFQVPQNYTKANCTYCNTREYTFSYKGCCFYFTKKKHTWNGCFQACAELYPCTYFYGPTPDILPVVTRNLNAIESLWVGVYRVGEGNWTSLDGGTFKVYQIFGSHCTYVSKFSTVPVSHHECSFLKPCLCVSQRSNS

SEQ ID NO: 18 - protein, 357 aa, Artificial Sequence
  Human gammaherpesvirus 4
MFSCKQHLSLGACVFCLGLLASTPFIWCFVFANLLSLEIFSPWQTHVYRLGFPTACLMAVLWTLVPAKHAVRAVTPAIMLNIASALIFFSLRVYSTSTWVSAPCLFLANLPLLCLWPRLAIEIVYICPAIHQRFFELGLLLACTIFALSVVSRALEVSAVFMSPFFIFLALGSGSLAGARRNQIYTSGLERRRSIFCARGDHSVASLKETLHKCPWDLLAISALTVLVVCVMIVLHVHAEVFFGLSRYLPLFLCGAMASGGLYLGHSSIIACVMATLCTLSSVVVYFLHETLGPLGKTVLFISIFVYYFSGVAALSAAMRYKLKKFVNGPLVHLRVVYMCCFVFTFCEYLLVTFIKS

SEQ ID NO: 19 - protein, 394 aa, Artificial Sequence
  Human alphaherpesvirus 1
MGGAAARLGAVILFVVIVGLHGVRGKYALVDASLKMADPNRFRGKDLPVLDQLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQPELAPEDPEDSALLEDPVGTVAPQIPPNWHIPSIQDAATPYHPPATPNNMGLIAGAVGGSLLAALVICGIVYWMRRHTQKAPKRIRLPHIREDDQPSSHQPLFY

SEQ ID NO: 20 - protein, 338 aa, Artificial Sequence
  Human alphaherpesvirus 1
MLAVRSLQHLSTVVLITAYGLVLVWYTVFGASPLHRCIYAVRPTGTNNDTALVWMKMNQTLLFLGAPTHPPNGGWRNHAHICYANLIAGRVVPFQVPPDAMNRRIMNVHEAVNCLETLWYTRVRLVVVGWFLYLAFVALHQRRCMFGVVSPAHKMVAPATYLLNYAGRIVSSVFLQYPYTKITRLLCELSVQRQNLVQLFETDPVTFLYHRPAIGVIVGCELMLRFVAVGLIVGTAFISRGACAITYPLFLTITTWCFVSTIGLTELYCILRRGPAPKNADKAAAPGRSKGLSGVCGRCCSIILSGIAVRLCYIAVVAGVVLVALHYEQEIQRRLFDV

SEQ ID NO: 21 - protein, 904 aa, Artificial Sequence
  Human alphaherpesvirus 1
MRQGAPARGRRWFVVWALLGLTLGVLVASAAPSSPGTPGVAAATQAANGGPATPAPPAPGAPPTGDPKPKKNKKPKPPKPPRPAGDNATVAAGHATLREHLRDIKAENTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYLANGGFLIAYQPLLSNTLAELYVREHLREQSRKPPNPTPPPPGASANASVERIKTTSSIEFARLQFTYNHIQRHVNDMLGRVAIAWCELQNHELTLWNEARKLNPNAIASATVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAAMFAGLGAFFEGMGDLGRAVGKVVMGIVGGVVSAVSGVSSFMSNPFGALAVGLLVLAGLAAAFFAFRYVMRLQSNPMKALYPLTTKELKNPTNPDASGEGEEGGDFDEAKLAEAREMIRYMALVSAMERTEHKAKKKGTSALLSAKVTDMVMRKRRNTNYTQVPNKDGDADEDDL

SEQ ID NO: 22 - protein, 223 aa, Artificial Sequence
  Human alphaherpesvirus 1
MGILGWVGLIAVGVLCVRGGLPSTEYVIRSRVAREVGDILKVPCVPLPSDDLDWRYETPSAINYALIDGIFLRYHCPGLDTVLWDRHAQKAYWVNPFLFVAGFLEDLSYPAFPANTQETETRLALYKEIRQALDSRKQAASHTPVKAGCVNFDYSRTRRCVGRQDLGPTNGTSGRTPVLPPDDEAGLQPKPLTTPPPIIATSDPTPRRDAATKSRRRRPHSRR

SEQ ID NO: 23 - protein, 838 aa, Artificial Sequence
  Human alphaherpesvirus 1
MGNGLWFVGVIILGVAWGQVHDWTEQTDPWFLDGLGMDRMYWRDTNTGRLWLPNTPDPQKPPRGFLAPPDELNLTTASLPLLRWYEERFCFVLVTTAEFPRDPGQLLYIPKTYLLGRPPNASLPAPTTVEPTAQPPPSVAPLKGLLHNPAASVLLRSRAWVTFSAVPDPEALTFPRGDNVATASHPSGPRDTPPPRPPVGARRHPTTELDITHLHNASTTWLATRGLLRSPGRYVYFSPSASTWPVGIWTTGELVLGCDAALVRARYGREFMGLVISMHDSPPVEVMVVPAGQTLDRVGDPADENPPGALPGPPGGPRYRVFVLGSLTRADNGSALDALRRVGGYPEEGTNYAQFLSRAYAEFFSGDAGAEQGPRPPLFWRLTGLLATSGFAFVNAAHANGAVCLSDLLGFLAHSRALAGLAARGAAGCAADSVFFNVSVLDPTARLQLEARLQHLVAEILEREQSLALHALGYQLAFVLDSPSAYDAVAPSAAHLIDALYAEFLGGRVLTTPVVHRALFYASAVLRQPFLAGVPSAVQRERARRSLLIASALCTSDVAAATNADLRTALARADHQKTLFWLPDHFSPCAASLRFDLDESVFILDALAQATRSETPVEVLAQQTHGLASTLTRWAHYNALIRAFVPEASHRCGGQSANVEPRILVPITHNASYVVTHSPLPRGIGYKLTGVDVRRPLFLTYLTATCEGSTRDIESKRLVRTQNQRDLGLVGAVFMRYTPAGEVMSVLLVDTDNTQQQIAAGPTEGAPSVFSSDVPSTALLLFPNGTVIHLLAFDTQPVAAIAPGFLAASALGVVMITAALAGILKVLRTSVPFFWRRE

SEQ ID NO: 24 - protein, 693 aa, Artificial Sequence
  Human alphaherpesvirus 1
MSAREPAGRRRRASTRPRASPVADEPAGDGVGFMGYLRAVFRGDDDSELEALEEMAGDEPPVRRRREGPRARRRRASEAPPTSHRRASRQRPGPDAARSQSVRGRLDDDDEVPRGPPQARQGGYLGPVDARAILGRVGGSRVAPSPLFLEELQYEEDDYPEAVGPEDGGGARSPPKVEVLEGRVPGPELRAAFPLDRLAPQVAVWDESVRSALALGHPAGFYPCPDSAFGLSRVGVMHFASPDNPAVFFRQTLQQGEALAWYITGDGILDLTDRRTKTSPAQAMSFLADAVVRLAINGWVCGTRLHAEARGSDLDDRAAELRRQFASLTALRPVGAAAVPLLSAGGLVSPQSGPDAAVFRSSLGSLLYWPGVRALLDRDCRVAARYAGRMTYLATGALLARFNPDAVRCVLTREAAFLGRVLDVLAVMAEQTVQWLSVVVGARLHPHVHHPAFADVAREELFRALPLGSPAVVGAEHEALGDTAARRLLANSGLNAVLGAAVYALHTALATVTLKYARACGDAHRRRDDAAATRAILAAGLVLQRLLGFADTVVACVTLAAFDGGFTAPEVGTYTPLRYACVLRATQPLYARTTPAKFWADVRAAAEHVDLRPASSAPRAPVSGTADPAFLLKDLEPFPPAPVSGGSVLGPRVRVVDIMSQFRKLLMGDEGAAALRAHVSGRRATGLGGPPRP

SEQ ID NO: 25 - protein, 673 aa, Artificial Sequence
  Severe acute respiratory syndrome coronavirus 2
SQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAR

(19) United States
(12) Patent Application Publication
(10) Pub. No.: US 2021/0317170 A1
(43) Pub. Date: Oct. 14, 2021

(54) COMPOSITIONS AND METHODS FOR DRUG DELIVERY AND TREATING VIRAL INFECTIONS

(71) Applicant: UT-Battelle, LLC, Oak Ridge, TN (US)

(72) Inventor: Joseph Christopher Ellis, Knoxville, TN (US)

(21) Appl. No.: 17/086,957

(22) Filed: Nov. 2, 2020

Related U.S. Application Data
(63) Continuation-in-part of application No. 16/848,397, filed on Apr. 14, 2020.

(51) Int. Cl. C07K 14/165; A61K 47/69; C12N 15/113; A61K 9/107; A61K 9/127

(52) U.S. Cl. CPC C07K 14/165; A61K 47/6911; A61K 9/0019; A61K 9/107; A61K 9/1271; C12N 15/113

(57) ABSTRACT

The present disclosure is directed to compositions and methods for targeted drug delivery that comprise a biocompatible framework carrying at least one drug and a viral surface protein, where the viral surface protein mediates entry into a target cell and is attached to an outer surface of the biocompatible framework in the drug carrier.

### CROSS REFERENCE TO RELATED APPLICATION

This application a continuation in part of U.S. patent application Ser. No. 16/848,397, filed Apr. 14, 2020, the entire contents of which are incorporated herein by reference.

### STATEMENT REGARDING FEDERALLY SPONSORED RESEARCH OR DEVELOPMENT

This invention was made with government support under Prime Contract No. DE-AC05-00OR22725 awarded by the U.S. Department of Energy. The government has certain rights in the invention.

### INCORPORATION BY REFERENCE OF SEQUENCE LISTING

The Sequence Listing in an ASCII text file, named as 38317Z_4613_1_SequenceListing.txt of 119 KB, created on Oct. 21, 2020, and submitted to the United States Patent and Trademark Office via EFS-Web, is incorporated herein by reference.

### FIELD OF DISCLOSURE

This invention relates to compositions and methods for delivery of therapeutic substances to tissues and cells expressing a receptor for viral surface protein (e.g., the ACE2 receptor for SARS-CoV2 (COVID-19) spike protein). The delivery compositions disclosed herein are generally composed of a drug-loaded carrier linked to a viral surface protein.

### BACKGROUND

Severe acute respiratory syndrome coronavirus 2 (SARS-CoV2 or COVID-19) is a novel coronavirus that is responsible for an ongoing global pandemic. There are currently no proven treatment options for the disease and as a result it is spreading rapidly and overwhelming hospitals globally. Numerous pharmaceutical companies, government entities, and non-profit organizations are searching for a new therapeutic agent to treat and cure COVID-19. RNA viruses such as influenza, HIV, HBC, and HCV are also challenging to treat. A large part of that challenge is developing a molecule that inhibits viral replication and can also enter the host (human) cell. In addition, another obstacle is off target effects of a potential drug that can cause toxicity or require elevated concentrations of the drug. This disclosure addresses both of those challenges.

### SUMMARY OF THE DESCRIPTION

An aspect of the disclosure is directed to a composition comprising a drug carrier, the drug carrier comprising a biocompatible framework carrying at least one drug and a viral surface protein, wherein the viral surface protein mediates entry into a target cell and is attached to an outer surface of the biocompatible framework in the drug carrier.

Another aspect of this disclosure is directed to a method for targeted delivery of a drug to cells expressing a receptor for a viral surface protein in a mammal, comprising administering to the mammal an effective amount of a composition comprising a drug carrier, the drug carrier comprising a biocompatible framework carrying at least one drug and a viral surface protein, wherein the viral surface protein mediates entry into the cells and is attached to an outer surface of the biocompatible framework in the drug carrier.

Another aspect of this disclosure is directed to a method of treating a viral infection in a mammal, comprising administering to the mammal an effective amount of a composition comprising a drug carrier, the drug carrier comprising a biocompatible framework carrying at least one antiviral agent and a viral surface protein, wherein the viral surface protein mediates entry into the cells and is attached to an outer surface of the biocompatible framework in the drug carrier.

In some embodiments, the at least one drug is encapsulated in, intercalated in, embedded in, absorbed to, or conjugated to the biocompatible framework in the drug carrier. In some embodiments, the at least one drug is attached to an outer surface of the biocompatible framework in the drug carrier.

In some embodiments, the biocompatible framework comprises a biocompatible polymer, a liposome, or a micelle.

In some embodiments, the viral surface protein selectively targets cells having a higher density of a receptor for the viral surface protein compared to other cells in a mammal.

In some embodiments, the viral surface protein is a viral spike protein. In some embodiments, the viral surface protein is selected from the group consisting of a surface protein from Human Immunodeficiency Virus (HIV), Hepatitis B Virus (HBV), Hepatitis C Virus (HBV), influenza virus, Epstein-Barr virus (EBV, Human gammaherpesvirus 4), herpes simplex virus (HSV-1, Human alphaherpesvirus), Severe acute respiratory syndrome-related coronavirus (SARS-CoV), Middle East Respiratory Syndrome Related Coronavirus (MERS), and Severe acute respiratory syndrome-related coronavirus 2 (SARS-CoV2 or COVID-19).

In some embodiments, the viral surface protein is the spike protein of Severe acute respiratory syndrome-related coronavirus 2 (SARS-CoV2), and the receptor is ACE-2. In some embodiments, the spike protein of SARS-CoV2 comprises an amino acid sequence selected from a sequence with at least 90% sequence identity to the amino acid sequence shown in SEQ ID NO: 1 or SEQ ID NO: 25. In some embodiments, the spike protein of SARS-CoV2 comprises an amino acid sequence selected from a sequence with at least 90% sequence identity to the amino acid sequence shown in SEQ ID NO: 2.

In some embodiments, the viral surface protein comprises an amino acid sequence selected from a sequence with at least 90% sequence identity to an amino acid sequence selected from SEQ ID NOs: 1-25, or a functional fragment thereof.

In some embodiments, the at least one drug comprises an antiviral agent.

In some embodiments, the at least one drug is a biological drug selected from the group consisting of a microRNA, a messenger RNA (mRNA), a protein, a deoxyribonucleic acid (DNA), a ribonucleic acid (RNA), an RNA interference (RNAi) construct, or a clustered regularly interspaced short palindromic repeats (CRISPR) construct.

In some embodiments, the composition is in a pharmaceutically acceptable carrier.

In some embodiments, the compositions of the instant disclosure are administered by a method selected from enteral, topical, pulmonary, and injection administration methods.

In some embodiments, wherein a viral infection primarily affects lungs (as in influenza, SARS-CoV, SARS-CoV2 (COVID-19) or MERS infections), the composition is administered by a pulmonary or an intravenous administration method.

In some embodiments, the mammal is a human. In some embodiments, the human is has a high risk of infection as the human suffers from diabetes, heart disease, a pulmonary disorder, or a combination thereof.

### DETAILED DESCRIPTION

### General Description

### Drug Carrier

The “drug carrier” (also “drug-loaded carrier”) of the instant disclosure includes a biocompatible framework that carries at least one drug. The biocompatible framework can have any structure that is suitable for carrying a drug. The drug can be carried (i.e., incorporated) in an inner portion, on an outer surface, or a combination thereof, of the biocompatible framework.

In some embodiments, the biocompatible framework possesses a substantially hollow interior portion surrounded by a biocompatible material, which may or may not be porous. In the foregoing embodiment, the drug can reside in the hollow interior portion, in which case the surrounding biocompatible material encapsulates the drug. If the encapsulating material is porous, the pores may be nanoporous, mesoporous, or macroporous, or a combination thereof, as long as the pores are small enough to maintain a substantial portion (e.g., at least 50, 60, 70, 80, 90, or 95%) or all of the encapsulated drug within the encapsulating material at least during the time the composition is being carried in the subject and until it binds to a target cell. Once docked at a target cell, the pores may serve to slowly release the drug.

In another embodiment, the biocompatible framework possesses a structure in which the drug can intercalate, i.e., the drug can be embedded in, absorbed to, or conjugated to the biocompatible framework. The framework in which the drug can intercalate may or may not include a hollow interior portion. Thus, in some embodiments, the drug may be encapsulated while also being intercalated in another portion of the biocompatible framework. Such intercalating structures for drug delivery are well known in the art.

In some embodiments, the biocompatible framework is constructed of a biocompatible polymer, which may have an organic or inorganic backbone. The encapsulating polymer can be, for example, a polyhydroxyacid biopolyester, polysaccharide, polyacrylate, polymethacrylate, polyalkyleneglycol, polyphosphazene, polyanhydride, polyacetal, poly(ortho esters), polyurea, polyurethane, polyamide, poly(amino acid), polyphosphoester, or a co-polymer thereof. Other polymer chemistries are possible, such as polycarbonates, polypyrroles, polyoxazoline, and polysiloxanes. A comprehensive review of these and other biocompatible polymers and their use in drug delivery is provided in G. Vilar, et al., Current Drug Delivery, vol. 9, no. 4, 2012, pp. 1-28, which is herein incorporated by reference in its entirety.

As used herein, the phrase “polyhydroxyacid biopolyester” (i.e., “biopolyester”) is meant to encompass all of those biocompatible polymers, as known in the art, that possess ester bonds, many of which are microbially produced or are known to be biodegradable. Two particular subclasses of biopolyesters considered herein are the poly(α-hydroxy acid)s and poly(hydroxyalkanoates). The poly(hydroxyalkanoates) generally refer to polyesters of non-α-hydroxy acids, such as polyesters of β-, γ-, δ-, and ε-hydroxy acids.

In some embodiments, the biocompatible framework comprises a polysaccharide. The polysaccharide may be based on, for example, dextran, dextran sulfate, hyaluronic acid, alginate, heparin, chondroitin sulfate, pectin, pullulan, amylose, a cyclodextrin, a chitosan (e.g., chitosan, carboxymethyl chitosan, glycol chitosan, N-trimethyl chitosan, N-triethyl chitosan), cellulose, carboxymethyl cellulose, or glucomannan, or a combination or co-polymer thereof. Such polymers and their use in drug delivery are well known in the art, as evidenced by, for example, Z. Liu, et al., “Polysaccharides-based nanoparticles as drug delivery systems”, Advanced Drug Delivery Reviews, vol. 60, no. 15, 2008, pp. 1650-1662; and Saravanakumar G. et al., Curr. Med. Chem., 19(19), 2012, pp. 3212-3229, all of which are herein incorporated by reference in their entirety.

In some embodiments, the biocompatible framework comprises a vinyl addition polymer. Some examples of such polymers include polyacrylic acid, polyacrylate salt, polymethacrylic acid, polymethacrylate salt, poly(methyl acrylate), poly(methyl acrylate), poly(ethyl acrylate), poly(methyl methacrylate), poly(2-hydroxyethyl acrylate), poly(2-hydroxyethyl methacrylate), polyvinyl alcohol, polyvinyl acetate, and polyacrylamides, including N-substituted versions thereof, such as poly(N-isopropylacrylamide), as well as combinations and co-polymers thereof. Such polymers and their use in drug delivery are well known in the art, as evidenced by, for example, Garay-Jimenez, J. C., et al., Bioorg. Med. Chem. Lett., 21(15), 2011, pp. 4589-4591; S. Benita, et al., Journal of Microencapsulation, vol. 2, no. 3, 1985, pp. 207-222; R. Kumar, et al., Frontiers of Chemical Science and Engineering, vol. 7, no. 1, 2013, pp. 116-122; Y. Zhang, et al., Polym. Chem., 3, 2012, pp. 2752-2759; Hsuie, G. H., et al., Biomaterials, 22(13), 2001, pp. 1763-1769; and Sutar, P. B., et al., J. Mater. Sci. Mater. Med., 19(6), 2008, pp. 2247-2253, all of which are herein incorporated by reference in their entirety.

In some embodiments, the biocompatible framework comprises a polyalkylene glycol. The polyalkylene glycol can be any of the polyalkylene glycols known in the art, such as polyethylene glycol, polypropylene glycol, or poly(trimethylene glycol), or a combination or co-polymer thereof. Such polymers and their use in drug delivery are well known in the art, as evidenced by, for example, K. Knop, et al., Angewandte Chemie, vol. 49, no. 36, 2010, pp. 6288-6308; A. Mero, et al., Journal of Bioactive and Compatible Polymers, May 2009, vol. 24 no. 3 220-234; R. B. Greenwald, PRHS, vol. 21, no. 2, 2002, pp. 113-121; S. Parveen, et al., Eur. J. Pharmacol., 670 (2-3), 2011, pp. 372-383; E. Locatelli, et al., Journal of Nanoparticle Research, 14:1316, November 2012; H. Ocal, et al., Colloid and Polymer Science, 291:5, 2013, pp. 1235-1245; and T. Ishihara, et al., International Journal of Pharmaceutics, 375:1-2, 2009, pp. 148-54, all of which are herein incorporated by reference in their entirety.

In some embodiments, the biocompatible framework comprises a polyphosphazene. As known in the art, polyphosphazenes are polymers that possess an inorganic backbone constructed of alternating phosphorus and nitrogen atoms, with variable side chains attached to the phosphorus atoms. The polyphosphazenes have the particular advantage of synthetic flexibility, ease of fabrication, and matrix permeability. Such polymers and their use in drug delivery are well known in the art, as evidenced by, for example, S. Lakshmi, et al., Advanced Drug Delivery Reviews, vol. 55, no. 4, 2003, pp. 467-482; I. Teasdale, et al., Polymers, 5, 2013, pp. 161-187; and Y. Lemmouchi, et al., Macromolecular Symposia, 123, 1997, pp. 103-112, all of which are herein incorporated by reference in their entirety.

In some embodiments, the biocompatible framework comprises a polyanhydride polymer, which may be saturated or unsaturated, and either aliphatic or aromatic. Such polymers and their use in drug delivery are well known in the art, as evidenced by, for example, C. T. Laurencin, et al., J. Orthop. Res., 11(2), 1993, pp. 256-262; J. P. Jain, et al., Expert Opin. Drug Deliv., 5(8), 2008, pp. 889-907; and H. B. Rosen, et al., Biomaterials, vol. 4, April 1983, pp. 131-133; all of which are herein incorporated by reference in their entirety.

In some embodiments, the biocompatible framework comprises a polyacetal. Polyacetal polymers and their use in drug delivery are well known in the art, as evidenced by, for example, J.-K. Kim et al., Int. J. Pharm., 401 (1-2), 2010, pp. 79-86; S. E. Paramonov, et al., Bioconjug. Chem., 19 (4), April 2008, pp. 911-919; and M. J. Vincent, et al., Journal of Drug Targeting, vol. 12, no. 8, September 2004, pp. 491-501; all of which are herein incorporated by reference in their entirety.

In some embodiments, the biocompatible framework comprises a poly(ortho ester). Poly(ortho ester) polymers and their use in drug delivery are well known in the art, as evidenced by, for example, U.S. Pat. No. 6,524,606, J. Heller, et al., Adv. Drug. Deliv. Rev., 54(7), 2002, pp. 1015-1039; J. Heller, et al., Biomacromolecules, 5(5), 2004, pp. 1625-1632; all of which are herein incorporated by reference in their entirety.

In some embodiments, the biocompatible framework comprises a polyurea. Polyurea polymers and their use in drug delivery are well known in the art, as evidenced by, for example, U.S. Pat. No. 8,529,880; W. He, et al., Advanced Functional Materials, vol. 22, no. 19, 2012, pp. 4023-4031; F. Xiang, et al., Macromolecules, 4611), 2013, pp. 4418-4425; G. Morral-Ruiz, et al., Polymer, 53, 2012, pp. 6072-6080; and P. Cass, et al., Acta. Biomater., 9(9), 2013, pp. 8299-8307; all of which are herein incorporated by reference in their entirety.

In some embodiments, the biocompatible framework comprises a polyamide. Polyamide polymers and their use in drug delivery are well known in the art, as evidenced by, for example, U.S. Pat. No. 8,277,841, I. Gachard, et al., “Drug delivery from nonpeptidic α-amino acid containing polyamides”, Polymer Bulletin, vol. 38, no. 4, April 1997, pp. 427-431; and D. Crespy, et al., Macromolecular Chemistry and Physics, vol. 208, no. 5, March 2007, pp. 457-466; all of which are herein incorporated by reference in their entirety.

In some embodiments, the biocompatible framework comprises a polyurethane.

Polyurethane polymers and their use in drug delivery are well known in the art, as evidenced by, for example, U.S. Pat. No. 8,529,880, J. Y. Cherng, et al., Int. J. Pharm., vol. 450, no. 1-2, June 2013, pp. 145-162; L. Zhou, et al., Macromolecules, 44(4), 2011, pp. 857-864; M. Mandru, et al., Central European Journal of Chemistry, April 2013, vol. 11, no. 4, pp 542-553; F. Borcan, et al., Chemistry Central Journal, 6:87, August 2012; S.-G. Kang, et al., Macromolecular Research, vol. 18, no. 7, July 2010, pp. 680-685; and R. S. Harisha, et al., J. Chem. Sci., vol. 122, no. 2, March 2010, pp. 209-216; all of which are herein incorporated by reference in their entirety.

In some embodiments, the biocompatible framework comprises a poly(amino acid), i.e., polypeptide. The poly(amino acid) can be derived from any known natural or unnatural amino acid. Some examples of poly(amino acids) include poly-γ-glutamic acid, polyaspartic acid, polyserine, polythreonine, polylysine, polyglutamine, polyasparagine, polyarginine, and polycysteine, as well as copolymers thereof. Poly(amino acid) polymers and their use in drug delivery are well known in the art, as evidenced by, for example, A. Lalatsa, J. Control Release, 161(2), 2012, pp. 523-36; S. R. Yoon, et al., J. Biomed. Mater. Res. A, 100 (8), August 2012, pp. 2027-2033; B. Tian, et al., J. Mater. Chem., 22, 2012, pp. 17404-17414; J. Ding, et al., Nanotechnology, vol. 22, no. 49, 2011; K. Osada, et al., Journal of the Royal Society Interface, vol. 6, no. suppl. 3, Jun. 6, 2009, S325-S339; and B. Manocha, et al., Crit. Rev. Biotechnol., 28(2), 2008, pp. 83-99; all of which are herein incorporated by reference in their entirety.

In some embodiments, the biocompatible framework comprises a polyphosphoester. Polyphosphoester polymers and their use in drug delivery are well known in the art, as evidenced by, for example, Z. Zhao, et al., “Polyphosphoesters in drug and gene delivery”, Adv. Drug Deliv. Rev., 55(4), 2003, pp. 483-499; and J. Zhou, et al., Adv. Healthc. Mater., 2013 Aug. 30, doi: 10.1002/adhm.201300235; all of which are herein incorporated by reference in their entirety.

In another embodiment, the biocompatible framework is a liposome. As well known in the art, a liposome has a lipid bilayer structure formed by the ordered assembly of amphiphilic molecules. In an aqueous environment, the liposome possesses a hydrophobic layer having inner and outer surfaces that are hydrophilic. Thus, if the drug is suitably hydrophilic, the drug may be encapsulated in an interior portion of the liposome or be attached to an outer surface thereof, whereas, if the drug is suitably hydrophobic, the drug may be intercalated within the hydrophobic layer of the liposome. The liposome can have any of the compositions well known in the art, such as a phosphatidylcholine phospholipid composition, phosphatidylethanolamine phospholipid composition, phosphatidylinositol phospholipid composition, or phosphatidylserine phospholipid composition. Liposome compositions and their use in drug delivery are well known in the art, as evidenced by, for example, U.S. Pat. Nos. 8,304,565; 8,329,213; U.S. Application Pub. No. 2008/113015; Medina 0. P., et al., Curr. Pharm. Des., 2004; 10(24):2981-9; Allen T. M., et al., Adv. Drug Deliv. Rev., 2013 January; 65(1):36-48; and W. Gao, et al., J. Mater. Chem. B, 2013; all of which are herein incorporated by reference in their entirety.

In another embodiment, the biocompatible framework is a micelle. As well known in the art, a micelle is distinct from a liposome in that it is not a bilayer structure and possesses a hydrophobic interior formed by the ordered interaction of amphiphilic molecules. Thus, a drug of sufficient hydrophobicity may be intercalated or encapsulated within the micellular structure, while a drug of sufficient hydrophilicity may be attached to the outer surface of the micelle. The micelle can be constructed of any of the numerous biocompatible compositions known in the art, such as a PEG-PLA or PEG-PCL composition. Micellular compositions and their use in drug delivery are well known in the art, as evidenced by, for example, U.S. Pat. Nos. 8,529,917; 8,367,113; T. Riley, et al., Colloids and Surfaces B: Biointerfaces, vol. 16, 1999, pp. 147-159; Croy and Kwon, Curr. Pharm. Design, 12:4669-4684 (2006); M.-C. Jones, et al, Eur. J. Pharmaceutics Biopharmaceutics, 48:101-111 (1999); Y. Yamamoto, et al., J. Control Release, 2001 Nov. 9; 77 (1-2): 27-38; and X. Yang et al., Journal of Biomedical Materials Research Part A, 2008, 86(1), pp. 48-60; all of which are herein incorporated by reference in their entirety.

In another embodiment, the biocompatible framework is a dendrimer. The dendrimer can be any of the dendrimers known in the art that can suitably carry a drug in a biological system, such as the well-known poly(amidoamine) (PAMAM) dendrimers, amino acid-based dendrimers, ester-containing (biodegradable) dendrimers, and glycodendrimers. Dendrimer compositions and their use in drug delivery are well known in the art, as evidenced by, for example, M. Ina, et al., Journal of Drug Delivery & Therapeutics, 2011, 1(2): pp. 70-74; S. Bai, et al., Crit. Rev. Ther. Drug Carrier Syst., 23(6), 2006, pp. 437-495; E. R. Gillies, et al., Drug Discovery Today, 10(1), 2005, pp. 35-43; and S. H. Medina, et al., Chem. Rev., 109, 2009, pp. 3141-3157, all of which are herein incorporated by reference in their entirety.

### Drug

In some embodiments, the drug comprises any biological or chemical substance useful in the treatment, prevention, or diagnosis of a disease or condition. As used herein, the term “drug” is also meant to encompass a “prodrug,” i.e., a substance that is altered or activated in the living subject to become an active drug. As used herein, the term “treatment” is meant to encompass curing, amelioration, remission, or management (e.g., prevention of progression), of the symptoms or etiology of a disease or condition. The term “treatment” also encompasses reducing the extent of an existing/ongoing disease or condition, as well as preventing the occurrence of a disease or condition. The drug can be, for example, antimicrobial (e.g., antibiotic, antiviral, anti-fungal, or anti-parasitic), analgesic (pain reliever), anti-inflammatory or anticancer.

In some embodiments, the antimicrobial drug comprises any drug that treats, prevents, or diagnoses an infection caused by a microbe, as provided in, for example, K. Lewis, Nature Reviews Drug Discovery, vol. 12, 2013, pp. 371-387; S. Crunkhorn, Nature Reviews Drug Discovery, 12, 2013, p. 99; E. D. Clercq, Nature Reviews Drug Discovery, 6, 2007, p. 941; M. A. Thompson, et al., JAMA, 308(4), 2012, pp. 387-402; the contents of which are herein incorporated in their entirety. The antibiotic drug can be, for example, a penicillin, cephalosporin, polymyxin, rifamycin, lipiarmycin, quinolone, sulfonamide, tetracycline, macrolide, lincosamide, cyclic lipopeptide, oxazolidinone, or glycylcycline, and combinations thereof. The antiviral drug can be, for example, acyclovir, famciclovir, valacyclovir, oseltamivir, zanamivir, abacavir, zidovudine, rimantadine, amantadine, lamivudine, nevirapine, efavirenz, emtricitabine, zalcitabine, tenofovir, rilpivirine, azidothymidine, or stavudine, and combinations thereof. The antifungal drug can be, for example, a polyene (e.g., filipin, nystatin, amphotericin B, natamycin) or an N-heterocycle (e.g., an imidazole, triazole, or thiazole antifungal drug). The antiparasitic drug can be, for example, thiabendazole, mebendazole, pyrantel pamoate, ivermectin, albendazole, niclosamide, praziquantel, rifampin, tinidazole, and metronidazole.

In some embodiments, the analgesic (pain reliever) drug comprises any drug that treats (i.e., relieves) or prevents pain, as provided in, for example, R. B. Silverman, et al., Nature Reviews Drug Discovery, 7, 711 (August 2008); and S. A. Schug, et al., CNS Drugs, 20(11), 2006, pp. 917-933; the contents of which are herein incorporated in their entirety. Some examples of analgesic drugs include paracetamol, acetaminophen, non-steroidal anti-inflammatory drugs (i.e., NSAIDs, such as aspirin, ibuprofen, naproxen, fenoprofen, and ketoprofen), ropivacaine, levobupivacaine, bupivacaine, opioids, pregabalin, morphine, fentanyl, sufentanil, clonidine, dexmedetomidine, epinephrine, baclofen, neostigmine, ketamine, midazolam and adenosine, mefenamic acid, tolfenamic acid, propofol, lorazepam, Cox-2 inhibitors (e.g., celecoxib, parecoxib, and etoricoxib), and the conotoxin ziconotide, and combinations thereof.

In some embodiments, the anti-inflammatory drug comprises any drug that treats or prevents inflammation, as provided in, for example, K. Peterson, et al., Final Update 4 Report, Oregon Health & Science University, November 2010; and S. L. Curry, et al., J. Am. Anim. Hosp. Assoc., 41 (5), September-October 2005, pp. 298-309; the contents of which are herein incorporated in their entirety. Some examples of anti-inflammatory drugs include aspirin, ketoprofen, meloxicam, etodolac, carprofen, deracoxib, and tepoxalin.

In some embodiments, the drug comprises a biological substance useful in the treatment, prevention, or diagnosis of a disease or condition. In some embodiments, the biological substance is selected from the group consisting of a microRNA, a messenger RNA (mRNA), a protein, a deoxyribonucleic acid (DNA), a ribonucleic acid (RNA), an RNA interference (RNAi) construct, or a clustered regularly interspaced short palindromic repeats (CRISPR) construct.

In some embodiments, a microRNA comprises a small non-coding RNAs that control gene expression by targeting complementary mRNA sequences. In some embodiments, a microRNA is used to treat diseases including, but not limited to cancer, neurodegenerative disease, immunology, and metabolic syndromes (see, e.g., Rupaimoole, R., & Slack, F. J. (2017). Nature Reviews Drug Discovery, 16(3), 203; Chen, C. et al., (2017). BioMed research international, 2017; Hanna, J. et al., (2019). Frontiers in genetics, 10, 478, all of which are herein incorporated by reference in their entirety).

In some embodiments, an mRNA comprises a single strand RNA molecule that is complementary to a strand of DNA that encodes a gene. In some embodiments, an mRNA is then translated into a protein in the cell that performs a function (or multiple functions). In some embodiments, diseases that are treated with the expression of an mRNA molecule include, but are not limited to, cancer, tissue damage, neurodegenerative disease, and heart disease (see, e.g., Dhaliwal, H. K. et al., (2020). Molecular Pharmaceutics. 17, 6, 1996-2005; Van Hoecke, L., & Roose, K. (2019). Journal of translational medicine, 17(1), 54, all of which are herein incorporated by reference in their entirety).

In some embodiments, a protein molecule comprises a polypeptide in the cell that performs a function (or multiple functions). In some embodiments, diseases that are treated with a protein molecule include, but are not limited to, cancer, tissue damage, neurodegenerative disease, and heart disease (see, e.g., Leader, B., Baca, Q. J., & Golan, D. E. (2008). Nature Reviews Drug Discovery, 7(1), 21-39, which is herein incorporated by reference in their entirety).

In some embodiments, DNA is introduced into the cell of a subject for gene therapy. In some embodiments, the subject is a mammalian. In some embodiments, the subject is an animal. In a specific embodiment, the subject is a human. In some embodiments, DNA is in a virus, a plasmid (circular), or present as a linear DNA molecule. In some embodiments, diseases that are treated with a DNA molecule include, but are not limited to, cancer, tissue damage, neurodegenerative disease, heart disease, genetic disorders such as cystic fibrosis, Huntington's disease (see, e.g., Saraswat, P. et al., (2009). Indian journal of pharmaceutical sciences, 71(5), 488, which is herein incorporated by reference in their entirety).

In some embodiments, an RNAi comprises a small non-coding RNAs that inhibits mRNA translation by targeting complementary mRNA sequences. In some embodiments an RNAi is used to treat diseases including, but not limited to cancer, neurodegenerative disease, immunology, and metabolic syndromes (see, e.g., Setten, R. L. et al., (2019). Nature Reviews Drug Discovery, 18(6), 421-446; Aagaard, L., & Rossi, J. J. (2007). Advanced drug delivery reviews, 59 (2-3), 75-86, all of which are herein incorporated by reference in their entirety).

In some embodiments, a CRISPR construct comprises an RNA-guided endonuclease comprising a nuclease, such as Cas9, and a guide RNA that directs cleavage of the DNA by hybridizing to a recognition site in the genomic DNA. CRISPR/Cas system is a method based on the bacterial type II CRISPR (clustered regularly interspaced short palindromic repeats)/Cas (CRISPR-associated) immune system. The CRISPR/Cas system allows targeted cleavage of genomic DNA guided by a customizable small noncoding RNA, resulting in gene modifications by both non-homologous end joining (NHEJ) and homology-directed repair (HDR) mechanisms. CRISPR-Cas and similar gene targeting systems are well known in the art with reagents and protocols readily available. Exemplary genome editing protocols are described in Jennifer Doudna, and Prashant Mali, “CRISPR-Cas: A Laboratory Manual” (2016) (CSHL Press, ISBN: 978-1-621821-30-4) and Ran, F. Ann, et al. (Nature Protocols (2013), 8 (11): 2281-2308, all of which are herein incorporated by reference in their entirety. In some embodiments, diseases that are treated with a CRISPR construct include, but are not limited to, cancer, tissue damage, neurodegenerative disease, heart disease, genetic disorders such as cystic fibrosis, Huntington's disease (see, e.g., Batzir, N. A., Tovin, A., & Hendel, A. (2017). Pediatric Endocrinology Reviews: PER, 14(4), 353-363; Yi, L., & Li, J. (2016). Biochimica et Biophysica Acta (BBA)-Reviews on Cancer, 1866(2), 197-207, all of which are herein incorporated by reference in their entirety).

### Viral Surface Protein

The phrase “viral surface protein,” as used herein, refers to a protein on the surface of a virus that mediates targeting, attachment and entry into a target cell. In some embodiments, the viral surface protein is a viral envelope protein. In some embodiments, the viral surface protein is a spike protein.

A “functional fragment” of a viral surface protein refers to a fragment or a part of the viral surface protein that is sufficient to mediate targeting, attachment and entry into a target cell. In some embodiments, a functional fragment comprises at least 15 amino acids, at least 20 amino acids, at least 25 amino acids, at least 30 amino acids, at least 35 amino acids, at least 40 amino acids, at least 45 amino acids, at least 50 amino acids, or longer.

In some embodiments, the viral surface protein is selected a surface protein from Human Immunodeficiency Virus (HIV), Hepatitis B Virus (HBV), Hepatitis C Virus (HBV), influenza virus, Epstein-Barr virus (EBV, Human gammaherpesvirus 4), herpes simplex virus (HSV-1, Human alphaherpesvirus), Severe acute respiratory syndrome-related coronavirus (SARS-CoV), Middle East Respiratory Syndrome Related Coronavirus (MERS), and Severe acute respiratory syndrome-related coronavirus 2 (SARS-CoV2 or COVID-19).

TABLE 1
Exemplary viral surface proteins

| | | SEQ ID |
|---|---|---|
| Virus | Surface Protein | NO: |
| SARS-CoV2 | Spike protein | 1 |
| SARS-CoV2 | Spike protein binding area | 2 |
| SARS-CoV | Spike protein | 3 |
| SARS-CoV | CTD1 binding domain | 4 |
| SARS-CoV | Spike protein binding area | 5 |
| MERS | Spike protein | 6 |
| HIV | gp120 envelope protein | 7 |
| HIV | gp120/gp41 envelope protein | 8 |
| Influenza A (H1N1) | Hemagglutinin (HA) protein | 9 |
| Influenza A (H1N1) | Neuraminidase (NA) protein | 10 |
| HBV | large envelope protein L-HNsAg | 11 |
| HCV | E1 protein | 12 |
| HCV | E2 protein | 13 |
| EBV | Glycoprotein 350 (gp350) | 14 |
| EBV | envelope glycoprotein H | 15 |
| EBV | envelope glycoprotein L | 16 |
| EBV | envelope glycoprotein 42 (gp42) | 17 |
| EBV | BMRF2 | 18 |
| HSV-1 | envelope glycoprotein B | 19 |
| HSV-1 | envelope glycoprotein K | 20 |
| HSV-1 | envelope glycoprotein B | 21 |
| HSV-1 | envelope glycoprotein L | 22 |
| HSV-1 | envelope glycoprotein H | 232 |
| HSV-1 | tegument protein VP13/14 | 24 |
| SARS-CoV2 | Spike protein | 25 |

### Compositions

An aspect of this disclosure is directed to a composition comprising a drug carrier, the drug carrier comprising a biocompatible framework carrying at least one drug and a viral surface protein, wherein the viral surface protein mediates entry into a target cell and is attached to an outer surface of the biocompatible framework in the drug carrier. In some embodiments, the composition comprises more than one type of viral surface protein. In some embodiments, the composition comprises at least two viral surface proteins as shown by SEQ ID NOs: 1-24, or fragments thereof. In some embodiments, different viral surface proteins mediate targeting and entry of the composition to different cell types.

In some embodiments. the at least one drug is encapsulated in, intercalated in, embedded in, absorbed to, or conjugated to the biocompatible framework in the drug carrier. In some embodiments, the at least one drug is attached to an outer surface of the biocompatible framework in the drug carrier.

In some embodiments, the viral surface protein is selected from a surface protein from Human Immunodeficiency Virus (HIV), Hepatitis B Virus (HBV), Hepatitis C Virus (HBV), influenza virus, Epstein-Barr virus (EBV, Human gammaherpesvirus 4), herpes simplex virus (HSV-1, Human alphaherpesvirus), Severe acute respiratory syndrome-related coronavirus (SARS-CoV), Middle East Respiratory Syndrome Related Coronavirus (MERS), or Severe acute respiratory syndrome-related coronavirus 2 (SARS-CoV2 or COVID-19). In some embodiments, the viral surface protein comprises an amino acid sequence with at least 90% sequence identity to an amino acid sequence selected from Table 1 (SEQ ID NOs: 1-25).

In some embodiments, the composition comprises a viral surface protein from SARS-CoV2. In some embodiments, the SARS-CoV2 viral surface protein is a SARS-CoV2 spike protein. In a specific embodiment, the SARS-CoV2 spike protein comprises an amino acid sequence with at least 90%, at least 92%, at least 95%, at least 98%, at least 99% or more sequence identity to the amino acid sequence shown in SEQ ID NO: 1, or a fragment thereof. In a specific embodiment, the SARS-CoV2 spike protein comprises an amino acid sequence with at least 90%, at least 92%, at least 95%, at least 98%, at least 99% or more sequence identity to the amino acid sequence shown in SEQ ID NO: 25, or a fragment thereof. In a specific embodiment, the fragment of SARS-CoV2 spike protein comprises an amino acid sequence with at least 90%, at least 92%, at least 95%, at least 98%, at least 99% or more sequence identity to the amino acid sequence shown in SEQ ID NO: 2.

As all compositions described herein are designed to be administered into living subjects, the entire compositions, including all components, should be biocompatible. The term “biocompatible,” as used herein, refers to the characteristic of certain substances to not induce a substantially adverse physiological response (e.g., acute immunological or toxicological response) in a subject being treated, wherein the term “substantially adverse physiological response” generally refers to a physiological response of such acuteness that the welfare of the subject or the efficacy of the treatment could be compromised, as could be determined by one skilled in the medical arts. Thus, a determination of biocompatibility is distinct from determining side effects, since side effects are generally an accepted aspect of any drug. As understood in the art, the term “biocompatible” is a relative term, since some degree of immune response and/or toxicity is common for most materials internalized into a subject, including those materials generally deemed biocompatible. In some embodiments, the term “biocompatible” indicates that the composition or a component thereof fails to elicit any detectable immunological or toxicological response from the subject being administered the composition.

In another aspect, the invention is directed to a pharmaceutical composition containing any one or more of the above-described compositions in a pharmaceutically acceptable carrier or excipient. The phrase “pharmaceutically acceptable carrier” or “pharmaceutically acceptable excipient,” as used herein, refers to a pharmaceutically acceptable material, composition or vehicle, such as a liquid (diluent or excipient) or solid filler. In the pharmaceutical composition, the compound is generally dispersed in the physiologically acceptable carrier, by either being mixed (e.g., in solid form with a solid carrier) or dissolved or emulsified in a liquid carrier. The carrier should be compatible with the other ingredients of the formulation and physiologically safe to the subject. Any of the carriers known in the art can be suitable herein depending on the mode of administration. Some examples of suitable carriers include gelatin, fatty acids (e.g., stearic acid) and salts thereof, talc, vegetable fats or oils, gums and glycols, starches, dextrans, and the like.

The pharmaceutical composition can also include one or more auxiliary agents, such as stabilizers, surfactants, salts, buffering agents, additives, or a combination thereof. The stabilizer can be, for example, an oligosaccharide (e.g., sucrose, trehalose, lactose, or a dextran), a sugar alcohol (e.g., mannitol), or a combination thereof. The surfactant can be any suitable surfactant including, for example, those containing polyalkylene oxide units (e.g., Tween 20, Tween 80, Pluronic F-68), which are typically included in amounts of from about 0.001% (w/v) to about 10% (w/v). The salt or buffering agent can be any suitable salt or buffering agent, such as, for example, sodium chloride, or sodium or potassium phosphate, respectively. Some examples of additives include, for example, glycerol, benzyl alcohol, and 1,1,1-trichloro-2-methyl-2-propanol (e.g., chloretone or chlorobutanol). If required, the pH of the solutions can be suitably adjusted by inclusion of a pH adjusting agent.

Compositions and formulations for parenteral, intrathecal, or intraventricular administration may include sterile aqueous solutions that may also contain buffers, diluents, and other suitable additives such as, but not limited to, penetration enhancers, carrier compounds, and other pharmaceutically acceptable carriers or excipients.

Compositions and formulations for oral administration include powders or granules, suspensions or solutions in water or non aqueous media, capsules, sachets or tablets. Thickeners, flavoring agents, diluents, emulsifiers, dispersing aids or binders may be desirable.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Pharmaceutical compositions of the present invention include, but are not limited to, solutions, emulsions, and liposome containing formulations. These compositions may be generated from a variety of components that include, but are not limited to, preformed liquids, self emulsifying solids, and self emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non aqueous or mixed media. Aqueous suspensions may further contain substances that increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature, these formulations vary in the components and the consistency of the final product.

The pharmaceutical composition may or may not also include one or more additional pharmaceutically active or auxiliary compounds outside the scope of the compositions of this disclosure. The additional active compound may, for example, suitably improve, augment, or otherwise suitably adjust the activity of the composition, or suitably adjust or diminish an undesired aspect of the composition, such as a side effect. In some embodiments, the one or more additional pharmaceutically active compounds may serve to treat any of the diseases described herein.

The pharmaceutical compositions of the present invention may additionally contain other adjunct or therapeutic components or agents conventionally found in pharmaceutical compositions. Thus, for example, the compositions may contain additional compatible pharmaceutically active materials such as, for example, antipruritics, astringents, local anesthetics or anti inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present disclosure. The formulations can be sterilized and, if desired, mixed with auxiliary agents, e.g., lubricants, preservatives, stabilizers, wetting agents, emulsifiers, or salts for influencing osmotic pressure, buffers, colorings, flavorings and/or aromatic substances and the like, as long as they do not deleteriously interact with components of the formulation.

The invention further provides a kit comprising a composition according to the present disclosure in a pharmaceutically acceptable carrier. The kit can include any of the components typically used in the administration and use of a pharmaceutical. Thus, the kit may include any apparatus components necessary in the administration of the pharmaceutical, such as, for example, a packaged pharmaceutically acceptable dose of the pharmaceutical, instructions for use of the pharmaceutical, and accessories for administration, such as a needle or pad, if applicable, and any additional therapeutic agents to be co-administered to a subject.

### Methods of Drug Delivery and Therapeutic Treatment

In another aspect, the disclosure is directed to methods for targeted delivery of a drug to cells expressing a receptor of a viral surface protein. In some embodiments, the methods of the disclosure target cells having a higher density of a receptor for the viral surface protein compared to other cells in a mammal. In some embodiments, the viral surface protein is from SARS-CoV2, the viral surface protein receptor is ACE2 and the target cells are lung epithelial cells. Such targeted drug delivery is useful for therapeutic treatment of diseases or conditions. In some embodiments, the disease is a viral infection.

In some embodiments, the method for targeted delivery of a drug comprises administering to the mammal an effective amount of a composition comprising a drug carrier, the drug carrier comprising a biocompatible framework carrying at least one drug and a viral surface protein, wherein the viral surface protein mediates entry into the cells and is attached to an outer surface of the biocompatible framework in the drug carrier.

Another aspect of the disclosure is directed to a method of treating a viral infection in a mammal, comprising administering to the mammal an effective amount of a composition comprising a drug carrier, the drug carrier comprising a biocompatible framework carrying at least one antiviral agent and a viral surface protein, wherein the viral surface protein mediates entry into the cells and is attached to an outer surface of the biocompatible framework in the drug carrier.

In some embodiments, the at least one drug is encapsulated in, intercalated in, embedded in, absorbed to, or conjugated to the biocompatible framework in the drug carrier. In some embodiments, the at least one drug is attached to an outer surface of the biocompatible framework in the drug carrier.

In some embodiments, the biocompatible framework comprises biocompatible polymer, a liposome, or a micelle.

In some embodiments, the viral surface protein is selected from the group consisting of a surface protein from Human Immunodeficiency Virus (HIV), Hepatitis B Virus (HBV), Hepatitis C Virus (HBV), influenza virus, Epstein-Barr virus (EBV, Human gammaherpesvirus 4), herpes simplex virus (HSV-1, Human alphaherpesvirus), Severe acute respiratory syndrome-related coronavirus (SARS-CoV), Middle East Respiratory Syndrome Related Coronavirus (MERS), and Severe acute respiratory syndrome-related coronavirus 2 (SARS-CoV2 or COVID-19).

In some embodiments, the viral surface protein is the spike protein of Severe acute respiratory syndrome-related coronavirus 2 (SARS-CoV2), and the receptor is ACE-2.

In some embodiments, the spike protein of SARS-CoV2 comprises an amino acid sequence selected from a sequence with at least 90% sequence identity to the amino acid sequence shown in SEQ ID NO: 1. In some embodiments, the spike protein of SARS-CoV2 comprises an amino acid sequence selected from a sequence with at least 90% sequence identity to the amino acid sequence shown in SEQ ID NO: 2. In some embodiments, the viral surface protein comprises an amino acid sequence selected from a sequence with at least 90% sequence identity to an amino acid sequence selected from SEQ ID NOs: 1-24, or a functional fragment thereof.

In some embodiments, the composition of the instant disclosure is targeted and docked at a target cell via the viral surface protein. Once the composition described herein is docked at a target cell, the drug can be released by any of several mechanisms. For example, if the biocompatible framework carrying the drug is biodegradable, the framework will slowly or quickly degrade to release the drug. Moreover, the biocompatible framework may be tailored to include biodegradable groups particularly susceptible to enzymes located in biological tissue, such as disulfide or ester groups. In other embodiments, the biocompatible framework may be suitably porous to slowly release the drug over time. In some embodiments, the biocompatible framework may be tailored to be sufficiently water soluble to dissolve over time, or may be composed of a pH-activated material that releases the drug under biological pH conditions, as well known in the art, e.g., V. Balamuralidhara, et al., 2011, “pH Sensitive Drug Delivery Systems: A Review”, American Journal of Drug Discovery and Development, 1: 24-48.

The composition according to the instant disclosure or pharmaceutical composition thereof of the present invention may be administered in a number of ways. Administration may be enteral (i.e., oral), topical (i.e., on the skin, including ophthalmic and to mucous membranes, including vaginal and rectal delivery), pulmonary (e.g., by inhalation or insufflation of powders or aerosols, including by nebulizer; intratracheal, intranasal, epidermal and transdermal), or by injection (e.g., intravenously or intramuscularly). Parenteral administration includes intravenous, intraarterial, subcutaneous, intraperitoneal or intramuscular injection or infusion; or intracranial, e.g., intrathecal or intraventricular, administration. For oral administration, liquid or solid oral formulations can be given. These include, for example, tablets, capsules, pills, troches, elixirs, suspensions, and syrups.

In some embodiments, wherein the viral infection primarily affects lungs, the composition is administered by a pulmonary or an intravenous administration method.

In some embodiments, the mammal is a human. In some embodiments, the human is has a high risk of infection as the human suffers from diabetes, heart disease, a pulmonary disorder, or a combination thereof. In some embodiments, a “pulmonary disorder” includes, but is not limited to, emphysema, asthma, pneumonia, tuberculosis, chronic obstructive pulmonary disorder (COPD) and lung cancer. In some embodiments, the human is an old human over 70 years of age.

Dosing is dependent on the severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. The administering physician can easily determine optimum dosages, dosing methodologies and repetition rates.

The composition according to the instant disclosure is administered in a pharmaceutically effective (i.e., treatment-effective) amount, which is an amount suitable for effecting an observable favorable change in the course of the disease or condition. Optimum dosages may vary depending on the relative potency of individual compounds, and can generally be estimated based on EC₅₀ or IC₅₀ values found to be effective in in vitro and in vivo animal models or based on the examples described herein. In general, dosage is from 0.01 μg to 100 g per kg of body weight, and may be given once or more daily, weekly, monthly, or yearly. In different embodiments, the composition according to the instant disclosure is administered at a dosage of precisely, about, at least, above, up to, or less than, for example, 5 mg, 10 mg, 20 mg, 30 mg, 40 mg, 50 mg, 60 mg, 70 mg, 80 mg, 90 mg, 100 mg, 150 mg, 200 mg, 250 mg, 300 mg, 350 mg, 400 mg, 450 mg, 500 mg, 600 mg, 700 mg, 800 mg, 900 mg, 1000 mg, 1200 mg, or 1500 mg per administration, wherein the compound can be administered by any suitable schedule, e.g., once daily, once weekly, twice daily, or twice weekly. The composition according to the instant disclosure can also be administered in a way which releases the compound into the subject in a controlled manner over time (i.e., as a controlled release formulation), by means well known in the art, such as by use of a time release capsule or time-releasing (e.g., slow dissolving) physical form of the compound. The treating physician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the therapy is administered in maintenance doses, ranging from 0.01 μg to 100 g per kg of body weight, once or more daily, for a suitable time period.

In some embodiments, the disease or condition being treated is an infection, wherein the infection is caused by a microbe, such as a bacteria, virus, fungus, or parasite. The bacterial infection can be a result of, for example, a bacterium of the Bacilli, Cocci, Spirochaetes, or Vibrio class. The viral infection can be a result of, for example, a rhino virus, an influenza virus, a chicken pox virus, a herpes virus (e.g., a herpes simplex virus, Epstein-Barr virus), a hepatitis virus (e.g., Hepatitis A virus, Hepatitis B virus, Hepatitis C virus), a retrovirus (e.g., HIV), or a coronavirus (e.g., SARS-CoV, MERS, SARS-CoV2).

What is claimed is:

1. A composition comprising a drug carrier, the drug carrier comprising a biocompatible framework carrying at least one drug and a viral surface protein, wherein the viral surface protein mediates entry into a target cell and is attached to an outer surface of the biocompatible framework.

2. The composition of claim 1, wherein the at least one drug is encapsulated in, intercalated in, embedded in, absorbed to, or conjugated to the biocompatible framework in the drug carrier.

3. The composition of claim 1, wherein the at least one drug is attached to an outer surface of the biocompatible framework.

4. The composition of claim 1, wherein the biocompatible framework comprises a biocompatible polymer, a liposome, or a micelle.

5. The composition of claim 1, wherein the viral surface protein selectively targets cells having a higher density of a receptor for the viral surface protein compared to other cells in a mammal.

6. The composition of claim 1, wherein the viral surface protein is a viral spike protein.

7. The composition of claim 1, wherein the viral surface protein is selected from the group consisting of a surface protein from Human Immunodeficiency Virus (HIV), Hepatitis B Virus (HBV), Hepatitis C Virus (HBV), influenza virus, Epstein-Barr virus (EBV, Human gammaherpesvirus 4), herpes simplex virus (HSV-1, Human alphaherpesvirus), Severe acute respiratory syndrome-related coronavirus (SARS-CoV), Middle East Respiratory Syndrome Related Coronavirus (MERS), and Severe acute respiratory syndrome-related coronavirus 2 (SARS-CoV2 or COVID-19).

8. The composition of claim 5, wherein the viral surface protein is the spike protein of Severe acute respiratory syndrome-related coronavirus 2 (SARS-CoV2), and the receptor is ACE-2.

9. The composition of claim 8, wherein the spike protein of SARS-CoV2 comprises an amino acid sequence selected from a sequence with at least 90% sequence identity to the amino acid sequence shown in SEQ ID NO: 1 or SEQ ID NO: 25.

10. The composition of claim 8, wherein the spike protein of SARS-CoV2 comprises an amino acid sequence selected from a sequence with at least 90% sequence identity to the amino acid sequence shown in SEQ ID NO: 2.

11. The composition of claim 1, the viral surface protein comprises an amino acid sequence selected from a sequence with at least 90% sequence identity to an amino acid sequence selected from SEQ ID NOs: 1-25, or a functional fragment thereof.

12. The composition of claim 1, wherein the at least one drug comprises an antiviral agent.

13. The composition of claim 1, wherein the at least one drug is a biological drug selected from the group consisting of a microRNA, a messenger RNA (mRNA), a protein, a deoxyribonucleic acid (DNA), a ribonucleic acid (RNA), an RNA interference (RNAi) construct, or a clustered regularly interspaced short palindromic repeats (CRISPR) construct.

14. A pharmaceutical composition comprising the composition of claim 1 in a pharmaceutically acceptable carrier.

15. A method for targeted delivery of a drug to cells expressing a receptor for a viral surface protein in a mammal, comprising administering to the mammal an effective amount of a composition comprising a drug carrier, the drug carrier comprising a biocompatible framework carrying at least one drug and a viral surface protein, wherein the viral surface protein mediates entry into the cells and is attached to an outer surface of the biocompatible framework.

16. The method of claim 15, wherein the at least one drug is encapsulated in, intercalated in, embedded in, absorbed to, or conjugated to the biocompatible framework.

17. The method of claim 15, wherein the at least one drug is attached to an outer surface of the biocompatible framework.

18. The method of claim 15, wherein the biocompatible framework comprises biocompatible polymer, a liposome, or a micelle.

19. The method of claim 15, wherein the viral surface protein selectively targets cells having a higher density of a receptor for the viral surface protein compared to other cells in a mammal.

20. The method of claim 15, wherein the viral surface protein is a viral spike protein.

21. The method of claim 15, wherein the viral surface protein is selected from the group consisting of a surface protein from Human Immunodeficiency Virus (HIV), Hepatitis B Virus (HBV), Hepatitis C Virus (HBV), influenza virus, Epstein-Barr virus (EBV, Human gammaherpesvirus 4), herpes simplex virus (HSV-1, Human alphaherpesvirus), Severe acute respiratory syndrome-related coronavirus (SARS-CoV), Middle East Respiratory Syndrome Related Coronavirus (MERS), and Severe acute respiratory syndrome-related coronavirus 2 (SARS-CoV2 or COVID-19).

22. The method of claim 15, wherein the viral surface protein is the spike protein of Severe acute respiratory syndrome-related coronavirus 2 (SARS-CoV2), and the receptor is ACE-2.

23. The method of claim 22, wherein the spike protein of SARS-CoV2 comprises an amino acid sequence selected from a sequence with at least 90% sequence identity to the amino acid sequence shown in SEQ ID NO: 1 or SEQ ID NO: 25.

24. The method of claim 22, wherein the spike protein of SARS-CoV2 comprises an amino acid sequence selected from a sequence with at least 90% sequence identity to the amino acid sequence shown in SEQ ID NO: 2.

25. The method of claim 15, the viral surface protein comprises an amino acid sequence selected from a sequence with at least 90% sequence identity to an amino acid sequence selected from SEQ ID NOs: 1-25, or a functional fragment thereof.

26. The method of claim 15, wherein the at least one drug comprises an antiviral agent.

27. The method of claim 15, wherein the at least one drug is a biological drug selected from the group consisting of a microRNA, a messenger RNA (mRNA), a protein, a deoxyribonucleic acid (DNA), a ribonucleic acid (RNA), an RNA interference (RNAi) construct, or a clustered regularly interspaced short palindromic repeats (CRISPR) construct.

28. The method of claim 15, wherein the composition is administered by a method selected from enteral, topical, pulmonary, and injection administration methods.

29. The method of claim 22, wherein the composition is administered by a pulmonary or an intravenous administration method.

30. A method of treating a viral infection in a mammal, comprising administering to the mammal an effective amount of a composition comprising a drug carrier, the drug carrier comprising a biocompatible framework carrying at least one antiviral agent and a viral surface protein, wherein the viral surface protein mediates entry into the cells and is attached to an outer surface of the biocompatible framework.

31. The method of claim 30, wherein the at least one antiviral agent is encapsulated in, intercalated in, embedded in, absorbed to, or conjugated to the biocompatible framework.

32. The method of claim 30, wherein the at least one antiviral agent is attached to an outer surface of the biocompatible framework.

33. The method of claim 30, wherein the biocompatible framework comprises a liposome, or a micelle.

34. The method of claim 30, wherein the viral surface protein selectively targets cells having a higher density of a receptor for the viral surface protein compared to other cells in a mammal.

35. The method of claim 30, wherein the viral surface protein is a viral spike protein.

36. The method of claim 30, wherein the viral surface protein is selected from the group consisting of a surface protein from Human Immunodeficiency Virus (HIV), Hepatitis B Virus (HBV), Hepatitis C Virus (HBV), influenza virus, Epstein-Barr virus (EBV, Human gammaherpesvirus 4), herpes simplex virus (HSV-1, Human alphaherpesvirus), Severe acute respiratory syndrome-related coronavirus (SARS-CoV), Middle East Respiratory Syndrome Related Coronavirus (MERS), and Severe acute respiratory syndrome-related coronavirus 2 (SARS-CoV2 or COVID-19).

37. The method of claim 30, wherein the viral surface protein is the spike protein of Severe acute respiratory syndrome-related coronavirus 2 (SARS-CoV2), and the receptor is ACE-2.

38. The method of claim 37, wherein the spike protein of SARS-CoV2 comprises an amino acid sequence selected from a sequence with at least 90% sequence identity to the amino acid sequence shown in SEQ ID NO: 1 or SEQ ID NO: 25.

39. The method of claim 37, wherein the spike protein of SARS-CoV2 comprises an amino acid sequence selected from a sequence with at least 90% sequence identity to the amino acid sequence shown in SEQ ID NO: 2.

40. The method of claim 30, the viral surface protein comprises an amino acid sequence selected from a sequence with at least 90% sequence identity to an amino acid sequence selected from SEQ ID NOs: 1-25, or a functional fragment thereof.

41. The method of claim 30, wherein the composition is administered by a method selected from enteral, topical, pulmonary, and injection administration methods.

42. The method of claim 37, wherein the composition is administered by a pulmonary or an intravenous administration method.

43. The method of claim 30, wherein the mammal is a human.

44. The method of claim 43, wherein the human is has a high risk of infection.

45. The method of claim 44, wherein the human suffers from diabetes, heart disease or a pulmonary disorder.
